(19) United States
(12) Patent Application Publication
(10) Pub. No.: US 2017/0143467 A1
(43) Pub. Date: May 25, 2017

(54) COVERED STENT AND PREPARATION METHOD THEREFOR

(71) Applicant: BCM Co., Ltd., Gyeonggi-do (KR)

(72) Inventor: Byung Cheol MYUNG, Gyeonggi-do (KR)

(21) Appl. No.: 15/319,902

(22) PCT Filed: Jul. 10, 2015
(86) PCT No.: PCT/KR2015/007167

§ 371 Date: Dec. 19, 2016

(30) Foreign Application Priority Data
Jul. 11, 2014 (KR) 10-2014-0087700

(51) Int. Cl. A61F 2/04; A61L 31/14; A61L 31/10; A61F 2/89; A61F 2/90

(52) U.S. Cl. CPC A61F 2/04; A61F 2/89; A61F 2/90; A61L 31/10; A61L 31/14; A61F 2230/0069; A61F 2240/001; A61F 2250/0004; A61F 2220/0075; A61L 2420/02; A61L 2420/08

(57) ABSTRACT

Disclosed is a covered stent having bending characteristics, capable of maintaining a bent state and preventing a lesion from invading into the covered stent by solving the problem of a conventional stent having bending characteristics. The present invention provides a stent, wherein pieces of PTFE tape made of the same material can be prevented from being separated from each other to be maintained only through partial adhesion on the inside and outside of a cylindrical stent, invasion of the lesion can be prevented since the state where an entire outer surface of the stent is wrapped with the PTFE tape can be maintained, and the bent state of the cylindrical stent can be freely maintained since the PTFE tape located on the outside and the cylindrical stent can maintain a free state with each other by allowing no PTFE tape at a part of the inside of the cylindrical stent.

### TECHNICAL FIELD

The present invention relates generally to a covered stent and a preparation method therefor. More particularly, the present invention relates to a covered stent and a preparation method therefor, wherein the covered stent is capable of widening a stenosed site by being implanted in the stenosed site caused by a lesion occurring in various organs such as the biliary tract, the esophagus, the respiratory tract and the ureter, or in a transjugular intrahepatic portosystemic shunt, is capable of maintaining a bent state in accordance with a shape of a lumen even when being implanted within the lumen in a bent state, and is capable of preventing a stent having a long length, such as an esophageal stent or an obesity-preventing stent to be implanted in the esophagus, from twisting.

### BACKGROUND ART

In general, stents are implanted in the internal organs so as to push the lesion site and widen the stenosed organ. Such a stent is configured with a hollow cylindrical body having a plurality of rhombic openings by weaving one or more shape-memory alloy wires in an overlapping manner. Recently, various kinds of stents having different shapes have been developed.

Such a stent tends to remain straight, so there is a problem in that when the stent is implanted within a meandering tubular organ in the human body, the organ is brought to be straightened.

In an effort to solve the above-mentioned problem, Korean Patent No. 10-0457629 disclosed a stent capable of being maintained in a bent state.

With reference to FIGS. 1 to 3, the stent configured with a rhombic opening 5 is formed by weaving different wires 1 and 2 alternatively to each other with an interlocking portion 3 and an intersection portion 4.

When the stent 6 is bent under the action of external force, the wires 1 and 2 travel respectively such that the rhombic opening 5 is reduced or increased in size, thus the stent is maintained in the bent state.

Accordingly, the stent 6 is maintained in the bent state in such a manner as to be implanted within meandering tubular organs in the human body.

However, the stent 6 is problematic in that an invasively growing lesion may grow through the rhombic opening 5 of the stent 6 when the stent is implanted in an internal organ where a growing lesion is present, thereby obstructing the movement of food, blood, various body fluids such as biliary fluid, etc. inside a passage of the stent 6.

In an effort to solve the above-mentioned problem, as shown in FIG. 4, there is provided a covered stent 8 coated with silicone or PTFE 7 on inner and outer surfaces of the stent, which has a different shape from the stent 6.

Such a covered stent 8 is advantageous in that the rhombic opening 5 is blocked by a coating layer of silicone or PTFE 7, so it is possible to prevent invasion of the lesion into the covered stent 8, and is thus mainly implanted in organs having a lesion site that continues to grow.

However, there is a contradictory problem in that the coating layer of silicone or PTFE 7 blocks the rhombic opening 5, and at the same time completely fixes wires 1 and 2 that comprise the rhombic opening 5, such that the wires 1 and 2 are prevented from traveling freely even when external force is applied, and thus the covered stent 8 cannot be maintained in the bent state.

Thus, the covered stent 8 has the same problem of being maintained in the bent state as that of a conventional stent that tends to be returned to the straight shape, whereas the covered stent is employed in the meandering internal organs.

In an effort to overcome such disadvantages, Korean Patent No. 10-1006990 has been disclosed.

With reference to FIG. 2, this prior art patent discloses a method in which a stent 6 is configured with a plurality of rhombic openings 5 that are formed by weaving different shape-memory alloy wires 1 and 2 alternatively with a plurality of interlocking portions 3 and a plurality of intersection portions 4 such that the rhombic openings 5 are changeable in size by external force, and which is subjected to heat treatment. Then, as shown in FIG. 5, tapes 10 of PTFE or a similar material are wound at sections M divided in a lengthwise direction of the stent 6 with an interval N so that the coating layer 12 of silicone or a similar material is prevented from being coated on the interlocking portions 3 and the intersection portions 4 comprised of the stent 6 wound with the tape 10, and the rhombic spaces 5. Thus, uncoated interlocking portions 3, intersection portions 4, and the rhombic spaces 5 can be freely changed in size by external force so as to be maintained in the bent state.

With reference to FIG. 6, the stent 6 having bending characteristics is prepared by: a process of forming the plurality of rhombic openings 5 that are formed by weaving different shape-memory alloy wires 1 and 2 alternatively with the plurality of interlocking portions 3 and the plurality of intersection portions 4 such that the rhombic openings 5 are changeable in size by external force, and which is subjected to heat treatment for storing a shape of the stent 6; a process of preparing a jig 70 in which a cylindrical rod 50 that has a same or similar diameter as an inner diameter of the stent 6 is coated with a coating solution 60 of silicone or a similar lubricous material and is dried; a process of fitting the stent 6 over the jig 70; a process of winding the tapes 10 of PTFE or a similar material at the sections M divided in the lengthwise direction of the stent 6 with the interval N on the interlocking portions 3 and the intersection portions 4 of the stent 6, and thereby forming the rhombic spaces 5; a process of coating the stent taped with the tapes 10, 6 over the jig 70 with the coating layer 12 of silicone or the similar material so that the taped sections of the stent 6 remain free relative to the coating layer 12; and a process of removing the stent 6 subjected to the coating from the jig 70 and drying the coating layer 12 coated on the stent 6.

However, such the stent 6 is problematic in that since an upper surface of the tape 10 of PTFE is coated with the coating layer 12 of silicone, the tape 10 and the coating layer 12 are separated from each other due to heterogeneity.

Moreover, an upper surface of the tape 10 of PTFE is coated with the coating layer 12 of silicone, so there is a problem in that although it is required that the stent be maintained in a bent state after being bent, it is difficult for the stent to be maintained in the bent state due to a property of returning to the original shape, which is possessed by the coating layer 12 of silicone.

In addition, since an inside of the stent is coated with the coating layer 12 of silicone, it is difficult for the stent to be maintained in the bent state after the stent coated with dried the coating layer is bent, due to the said property of returning to the original shape, which is possessed by the coating layer 12 of silicone.

Furthermore, due to this reason, when the stent is bent at a certain angle or more, twisting of the stent occurs due to the coating layer 12 of silicone on the inside and outside of the stent, so it is difficult for the stent to be returned to the original shape, namely the straight shape, when the stent is required to be returned to the original shape after being bent. In order to solve these problems, as shown in FIG. 7, there has been an attempt to expose a part of an outer portion of the stent to the outside by removing the tape 10 of PTFE before the coating layer 12 of silicone coated on the upper surface of the tape 10 of PTFE is dried.

However, in this case, since the outside of the stent is exposed, the lesion grows into the stent through the rhombic openings 5 when the stent is implanted within a lumen of the human body. Thus, there is a further problem in that the growing lesion forces the coating layer 12 of silicone on the inside of the stent toward the inside of the passage of the stent, and thus the coating layer 12 blocks the passage of the stent.

### DISCLOSURE

### Technical Problem

Accordingly, the present invention has been made keeping in mind the above problems occurring in the related art, and the present invention is intended to propose a covered stent capable of being maintained in a bent state and preventing invasion of the lesion into the covered stent of the present invention by solving a problem of a conventional stent having bending characteristics.

### Technical Solution

In order to achieve the above object, according to one aspect of the present invention, there is provided a covered stent, wherein pieces of PTFE tape made of the same material can be maintained in a bonded state with each other through only a partial bonding on the inside and outside of a cylindrical stent, so it is possible to prevent the pieces of PTFE tape from being separated from each other. Due to this, the PTFE tape can be kept in a state of covering an entire outer surface of the cylindrical stent so it is possible to prevent invasion of a lesion into the cylindrical stent, moreover, the PTFE tape is allowed not to be provided at a part of the inside of the cylindrical stent so that the PTFE tape provided on the outside of the cylindrical stent and the cylindrical stent can remain free with respect to each other, and thus the cylindrical stent can be freely maintained in the bent state.

### Advantageous Effects

According to the present invention having the above-described characteristics, the present invention maintains excellent bending characteristics so it is possible to be implanted within a bent lumen in accordance with the degree of bending of the lumen. Moreover, since a PTFE tape covers on an entire outer surface of a cylindrical stent, it is possible to prevent invasion of a lesion such as growing cancerous cells inside a covered stent of the present invention even though the covered stent is implanted within any part of a lumen. In addition, pieces of PTFE tape made of the same material can be maintained in a bonded state with each other through only a partial bonding on the inside and outside of the cylindrical stent such that it is possible to prevent the pieces of PTFE tape from being separated from each other. Due to this, the PTFE tape can be kept in a state of covering the entire outer surface of the cylindrical stent.

### DESCRIPTION OF DRAWINGS

FIG. 1 is a view showing a configuration of a conventional stent.

FIG. 2 is a partially enlarged view of part A shown in FIG. 1.

FIG. 3 is a partially enlarged view of part B shown in FIG. 1.

FIG. 4 is a cross-sectional view showing a covered stent in the related art.

FIG. 5 is a view showing a configuration of a stent having bending characteristics in the related art.

FIG. 6 is a view showing a process of preparing the stent having bending characteristics in the related art. FIG. 7 is a view showing a configuration of the stent having bending characteristics in the related art.

FIG. 8 is a view showing a process of preparing a stent having bending characteristics of the present invention.

FIG. 9 is a partial cross-sectional view showing an inside of the covered stent of the present invention.

FIG. 10 is a view showing a configuration of the covered stent having bending characteristics of the present invention.

FIGS. 11 and 12 are views showing another embodiment of the present invention.

FIGS. 13 to 17 are photographs showing bending characteristics of an actual product according to the present invention.

FIGS. 18 to 22 are photographs showing bending characteristics of an actual product according to another embodiment of the present invention.

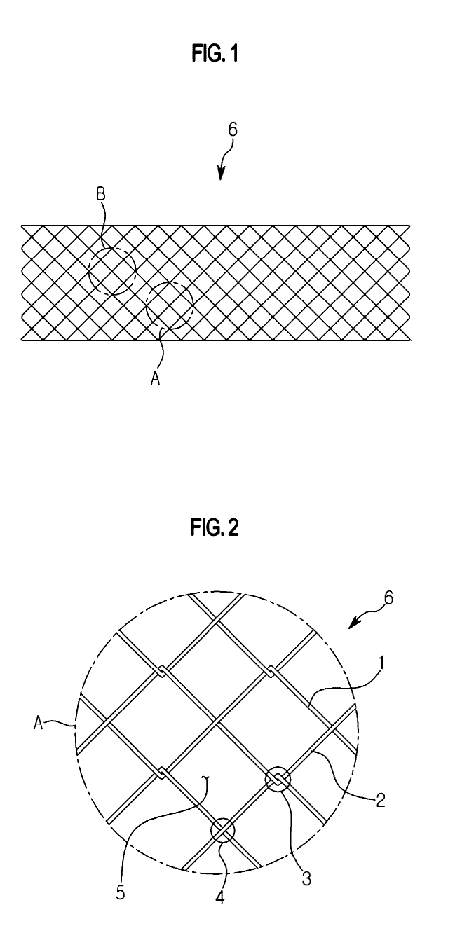

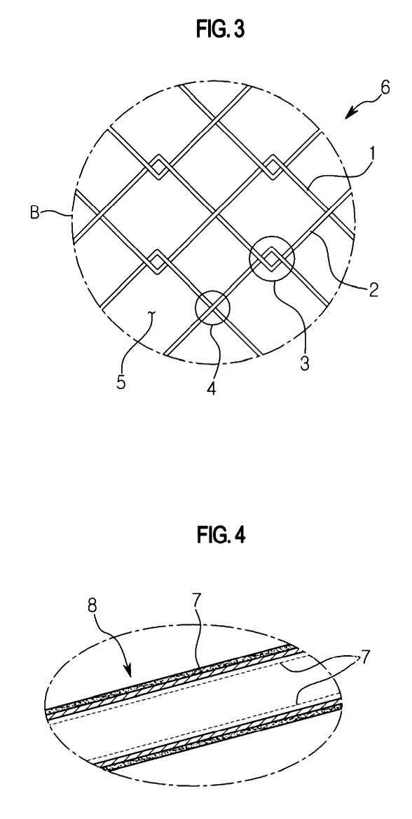

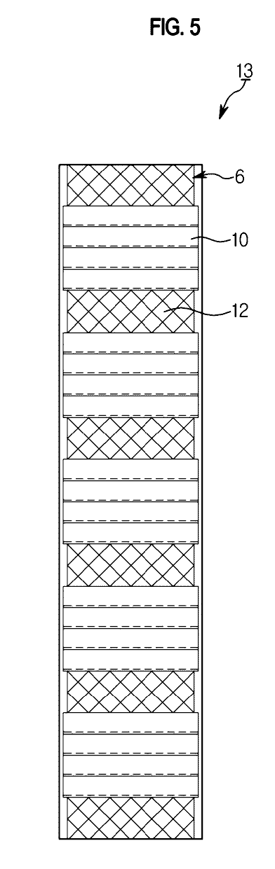

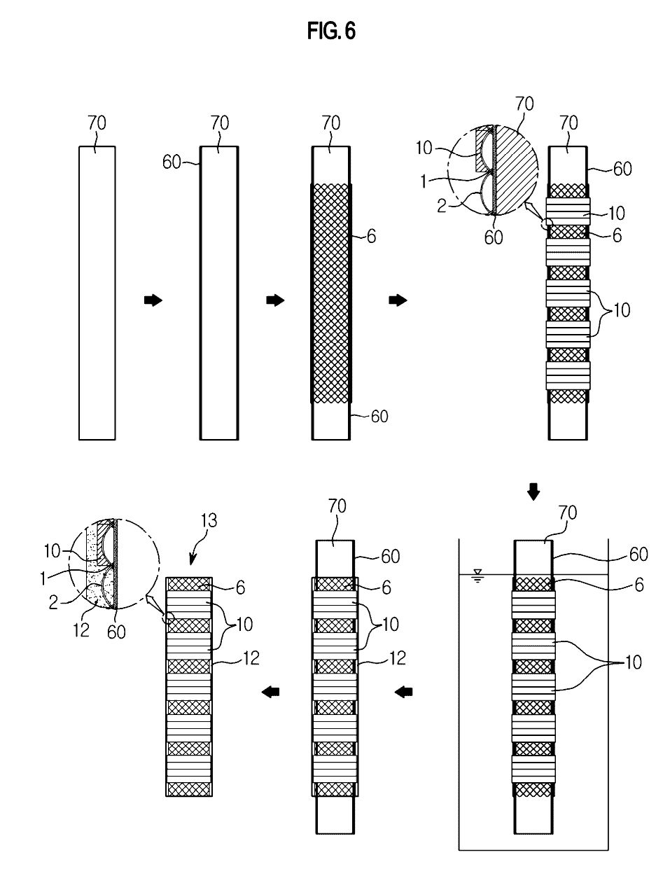

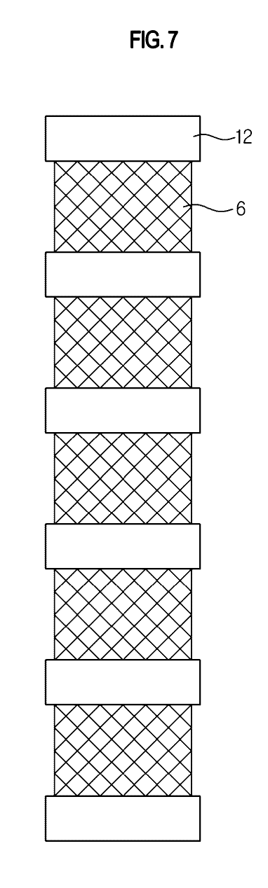

### BEST MODE

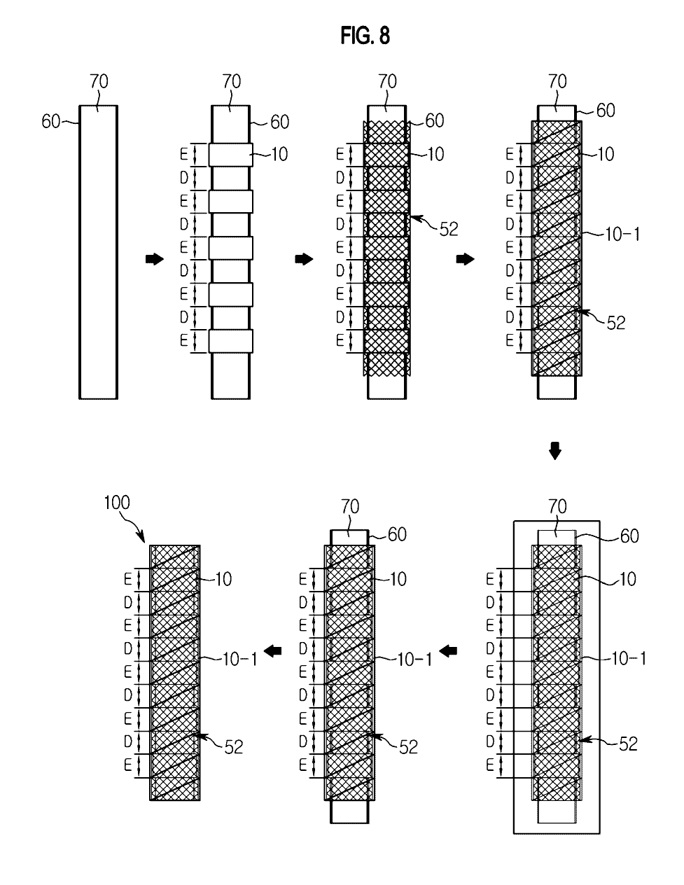

Hereinafter, the present invention will be described in detail with reference to the accompanying drawings. With reference to FIG. 8, a cylindrical rod 50 having a same or similar diameter as an inner diameter of a cylindrical stent (hereinafter referred to as cylindrical stent 52 that will be manufactured as a covered stent having bending characteristics of the present invention is coated with a coating solution of silicone or a similar lubricous material on a surface thereof and is dried to prepare a jig 70.

A process of winding PTFE tape 10 on the jig 70 at sections E divided in a lengthwise direction of the jig 70 with intervals D is carried out.

In this state, a process of fitting the cylindrical stent 52 having a plurality of rhombic openings 5 that are formed by weaving different shape-memory alloy wires 1 and 2 alternatively with a plurality of interlocking portions 3 and a plurality of intersection portions 4 such that the rhombic openings 5 are changeable in size by external force, over the jig 70 taped with the PTFE tape 10 at the sections E is carried out.

In this state, a process of winding a PTFE tape 10-1 on an entire outer surface of the cylindrical stent 52 is carried out.

After completion of the process of winding the PTFE tape 10-1 on the entire outer surface of the stent 52, heat and pressure are applied by using a heating and pressurizing device. By this process, the PTFE tape 10 provided on an inside of the cylindrical stent 52 and the PTFE tape 10-1 provided on an outside of the cylindrical stent 52 are bonded to each other through the rhombic openings 5 that comprise cylindrical stent 52, whereas the PTFE tape 10-1 provided at the intervals D on the outside of the cylindrical stent 52 remains free relative to the cylindrical stent 52 because no PTFE tape 10 is provided at the intervals D to be bonded to the PTFE tape 10-1.

In other words, at the sections E, the PTFE tape 10-1 provided on the outside of the cylindrical stent 52 and the PTFE tape 10 provided on the inside of the cylindrical stent 52 remain bonded to each other with the cylindrical stent 52 interposed therebetween. At the intervals D, the PTFE tape 10-1 provided on the outside of the cylindrical stent 52 remains free relative to the cylindrical stent 52.

A covered stent 100 having bending characteristics of the present invention is prepared by the above processes and is removed from the heating and pressurizing device, whereafter the covered stent 100 having bending characteristics of the present invention is removed from the jig 70. Here, since the outer surface of the cylindrical rod 50 is coated with the coating solution 60 of silicone or the similar lubricous material, it is possible to easily separate the covered stent 100 from the jig 70.

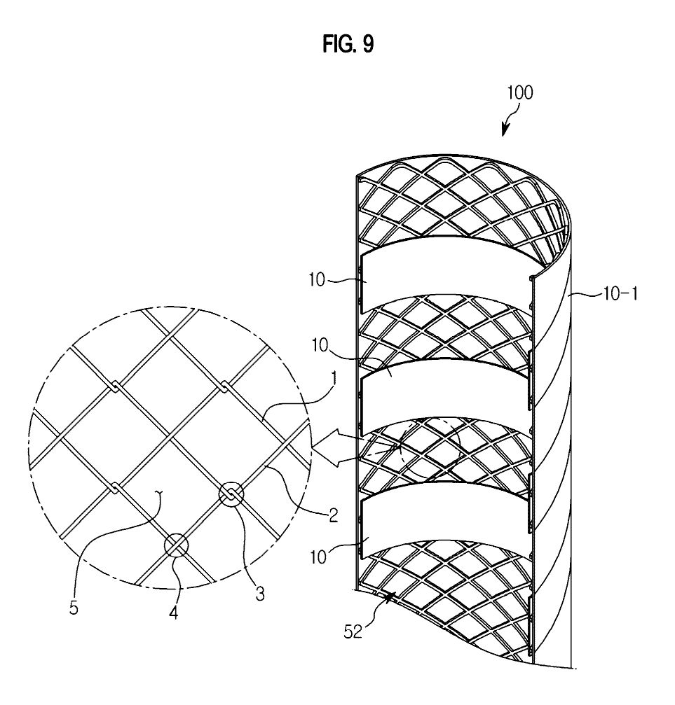

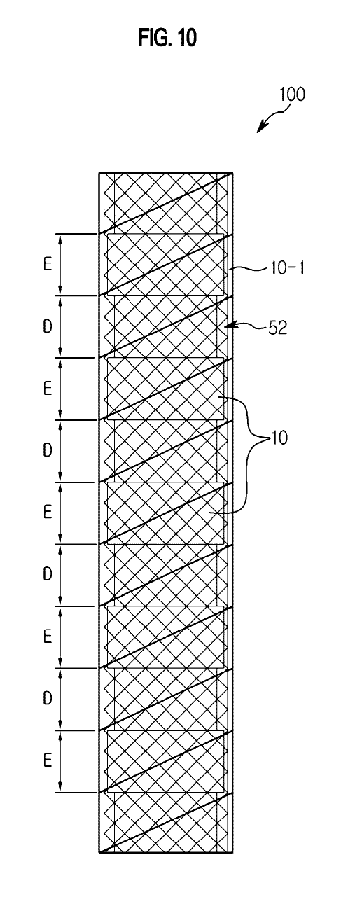

With reference to FIGS. 9 to 10, the PTFE tape 10 remains bonded to the PTFE tape 10-1 at the sections E on the inside of the cylindrical stent 52 with the intervals D, whereas no PTFE tape 10 is provided at the intervals D on the inside of the cylindrical stent 52, such that the PTFE tape 10-1 provided on the outside of the cylindrical stent 52 has no PTFE tape 10 to be bonded thereto, thereby remaining free with respect to the cylindrical stent 52. Thus, the rhombic openings 5 formed by weaving wires with the interlocking portions 3 and the intersection portions 4 at the intervals D of the cylindrical stent 52 can be changeable in size by external force. Thus, when the covered stent 100 having bending characteristics of the present invention is bent in one direction, the rhombic openings 5 located on a bent side are reduced in size in such a manner as to be maintained in a bent state.

As a result, it can be seen that the covered stent 100 having the bending characteristics of the present invention is capable of being maintained in the bent state at the intervals D.

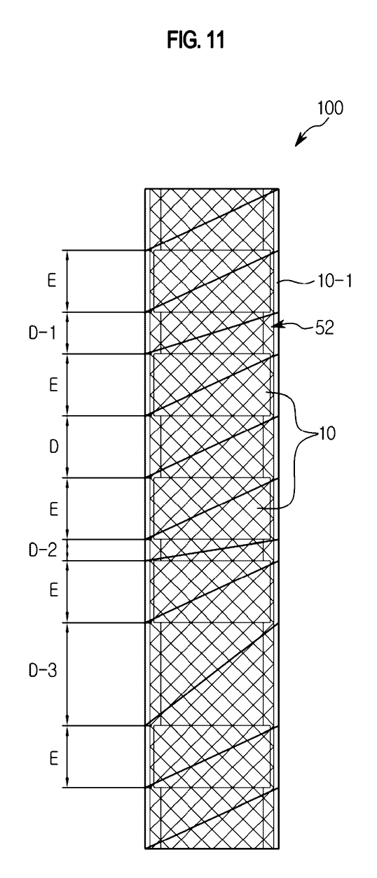

Here, the intervals D may be increased or reduced in number and width as occasion demands, and may be varied to have different widths D1, D2, and D3 as shown in FIG. 11.

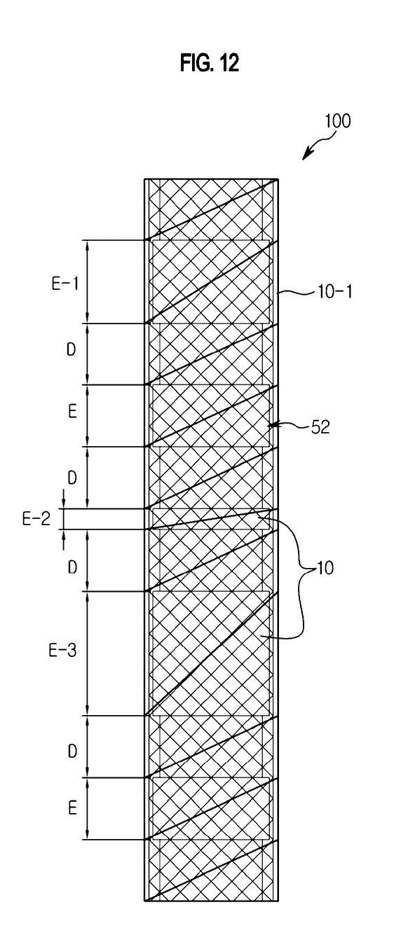

Likewise, the sections E may be increased or reduced in number and width as occasion demands, and may be varied to have different widths E1, E2, and E3 as shown in FIG. 12.

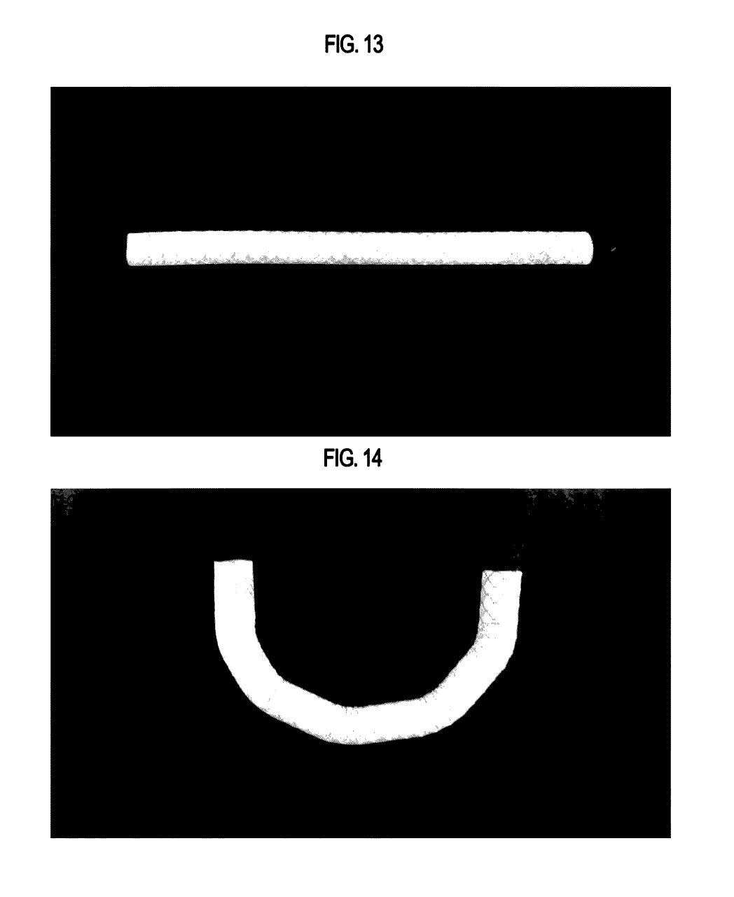

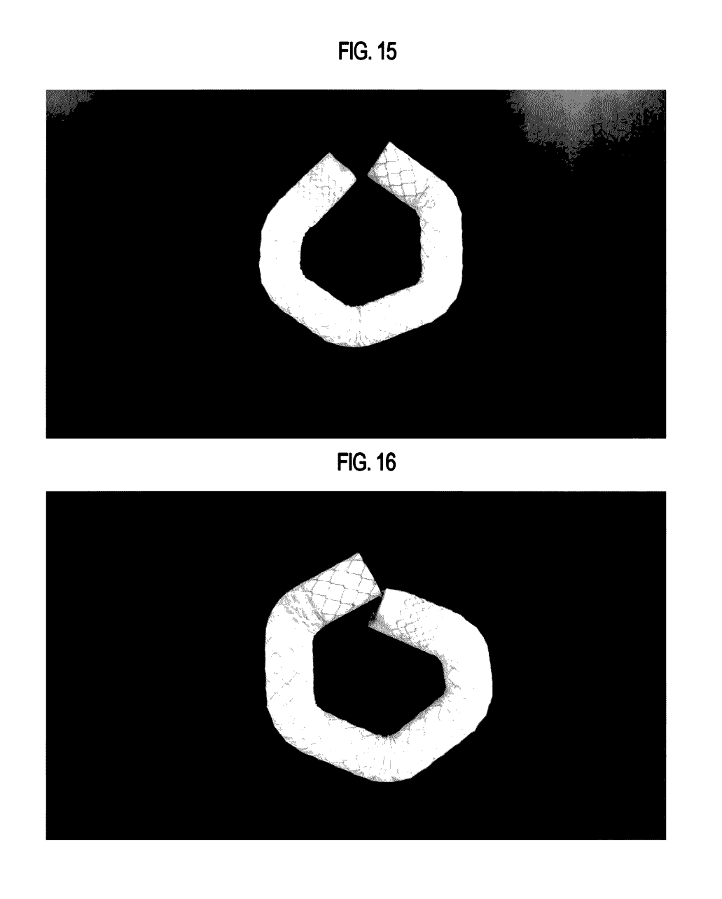

With reference to FIGS. 13 to 16, it can be seen that the present invention is in a straight state in FIG. 13, is maintained in a slightly bent state in FIG. 14, is maintained in a more bent state in FIG. 15 than in FIG. 14, namely is further bent from a semicircle, and is maintained in a bent state when both ends are bent until they almost come into contact with each other in FIG. 16.

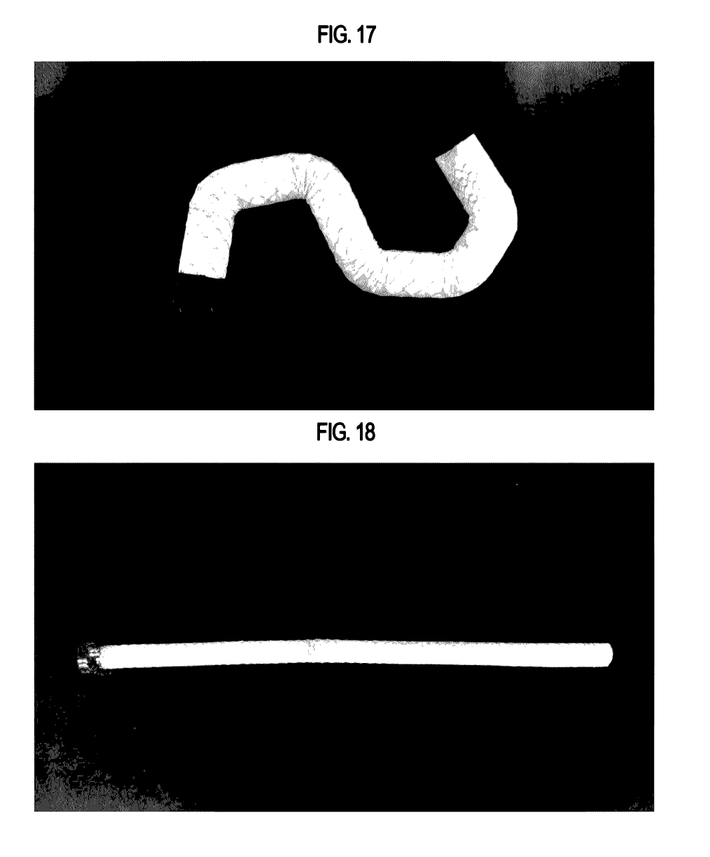

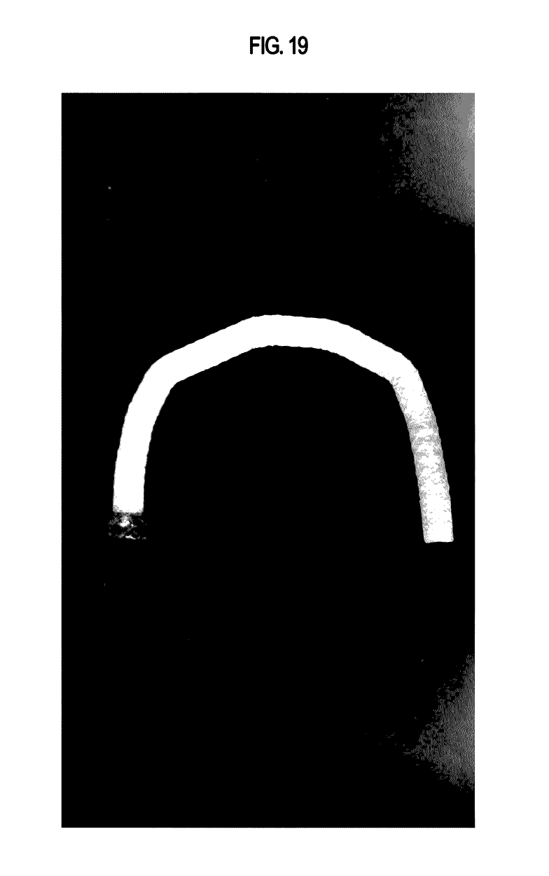

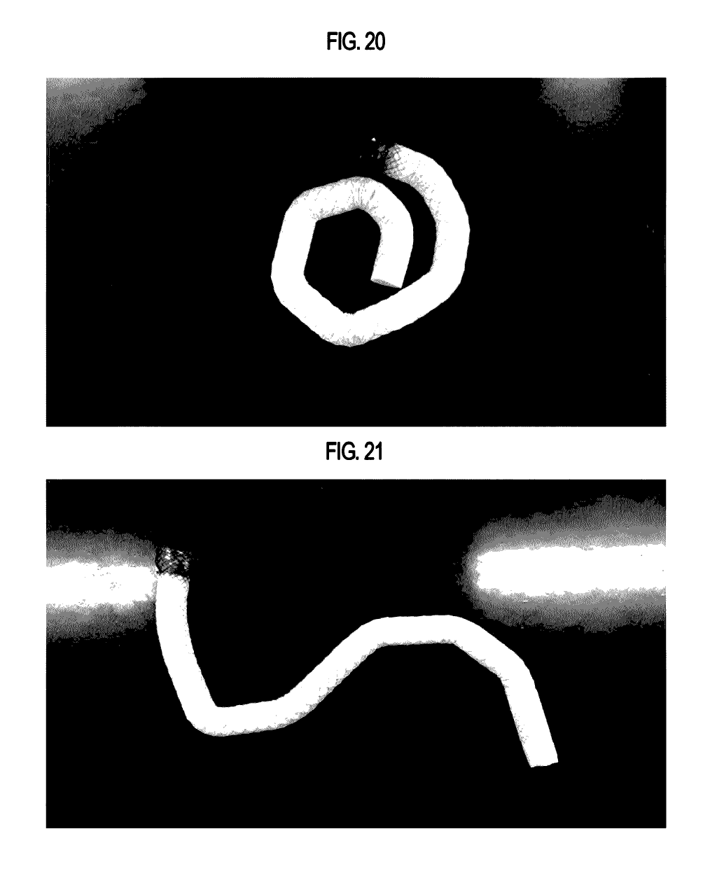

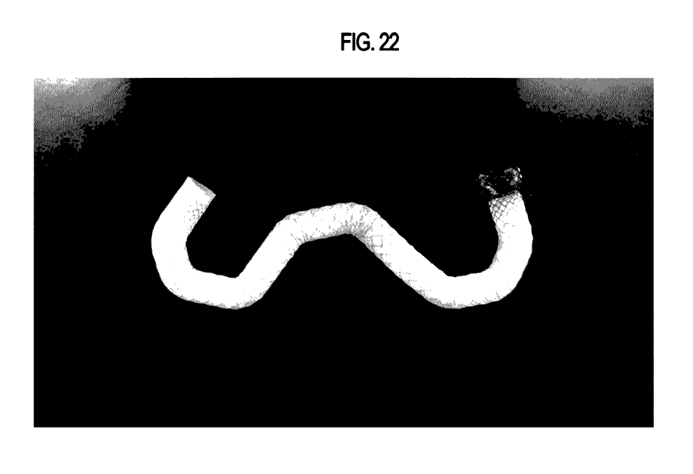

Further, with reference to FIG. 17, it can be seen that the present invention is maintained in a bent state when being bent in an S-shape. Thus, it is indicated that the covered stent having bending characteristics of the present invention can be maintained in the bent state although it is a stent having a PTFE tape covered thereon.

Moreover, with reference to FIG. 18, the present invention can be applied to an expandable stent having an expanded inlet at an end, and can be also applied to an expandable stent having expanded inlet and outlet at both ends.

As described above, according to the present invention, it can be understood that the present invention is a stent that maintains excellent bending characteristics, and thereby can be implanted within a bent lumen in accordance with the degree of bending of the lumen.

In addition, since the PTFE tape 10-1 is provided on the entire outer surface of the cylindrical stent 52, it is possible to prevent invasion of a lesion such as growing cancerous cells inside the covered stent 100 of the present invention even though the present invention is implanted within any part of a lumen, thereby being capable of performing intrinsic functions of a stent.

Thus, according to the present invention, pieces of PTFE tape made of the same material can remain bonded to each other through only a partial bonding on the inside and outside of the cylindrical stent such that it is possible to prevent the pieces of PTFE tape from being separated from each other. Due to this, the PTFE tape can be kept in a state of covering the entire outer surface of the cylindrical stent, such that it is possible to prevent invasion of the lesion into the cylindrical stent. Moreover, the PTFE tape is allowed not to be provided at a part of the inside of the cylindrical stent so that the PTFE tape provided on the outside of the cylindrical stent and the cylindrical stent can remain free relative to each other, and thus the cylindrical stent can be freely maintained in the bent state.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

### DESCRIPTION OF THE REFERENCE NUMERALS IN THE DRAWINGS

| | |
|---|---|
| 1, 2: wire | 3: interlocking portion |
| 4: intersection portion | 5: rhombic opening |
| 10, 10-1: PTFE tape | 52: cylindrical stent |
| 60: coating solution | 70: jig |
| 100: covered stent | |

1. A covered stent having bending characteristics, the covered stent being prepared by using a cylindrical stent, wherein a PTFE tape is provided at a section with an interval on an inside of the cylindrical stent such that the PTFE tape and a PTFE tape that is provided on an outside of the cylindrical stent remain bonded to each other, whereas the cylindrical stent remains free at the interval at which no PTFE tape is provided on the inside of the cylindrical stent, so rhombic openings of the cylindrical stent that are formed by weaving wires alternatively with a plurality of interlocking portions and a plurality of intersection portions are changeable in size by external force.

2. The covered stent of claim 1, wherein the interval at which no PTFE tape is provided on the inside of the cylindrical stent is provided in plural.

3. The covered stent of claim 1, wherein when providing the PTFE tape, the interval at which no PTFE tape is provided on the inside of the cylindrical stent is increased or reduced in width.

4. The covered stent of claim 1, wherein when a plurality of intervals at which no PTFE tape is provided on the inside of the cylindrical stent is provided, the intervals are different from each other in width.

5. The covered stent of claim 1, wherein the section at which the PTFE tape is provided on the inside of the cylindrical stent is provided in plural.

6. The covered stent of claim 1, wherein when mounting the PTFE tape, the section at which the PTFE tape is provided on the inside of the cylindrical stent is increased or reduced in width.

7. The covered stent of claim 1, wherein when a plurality of sections at which the PTFE tape is provided on the inside of the cylindrical stent is provided, the sections are different from each other in width.

8. A method of preparing a covered stent having bending characteristics, the covered stent being prepared by using a jig in which a cylindrical rod that has a same or similar diameter as an inner diameter of the cylindrical stent having rhombic openings that are formed by weaving shape-memory alloy wires alternatively with a plurality of interlocking portions and a plurality of intersection portions such that the rhombic openings are changeable in size by external force, is coated with a coating solution of silicone or a similar lubricous material on a surface thereof and is dried, the method comprising:
winding PTFE tape on the jig at sections divided in a lengthwise direction of the jig with an interval;
fitting the cylindrical stent over the jig taped with the PTFE tapes at the sections;
winding a PTFE tape on an entire outer surface of the cylindrical stent fitted over the jig;
applying heat and pressure by using a heating and pressurizing device after completion of the winding the PTFE tape on the entire outer surface of the cylindrical stent;
allowing the PTFE tape provided at the interval on an outside of the cylindrical stent to remain free relative to the cylindrical stent because no PTFE tape is provided at the interval to be bonded to the PTFE tape, whereas the PTFE tape provided on an inside of the cylindrical stent and the PTFE tape provided on the outside of the cylindrical stent are bonded to each other through the rhombic openings of the cylindrical stent; and
removing the jig from the cylindrical stent.

9. The method of claim 8, wherein the interval at which no PTFE tape is provided on the inside of the cylindrical stent is provided in plural.

10. The method of claim 8, wherein the interval at which no PTFE tape is provided on the inside of the cylindrical stent is increased or reduced in width.

11. The method of claim 8, wherein when a plurality of the intervals at which no PTFE tape is provided on the inside of the cylindrical stent is provided, the intervals are different from each other in width.

12. The covered stent of claim 2, wherein when providing the PTFE tape, the interval at which no PTFE tape is provided on the inside of the cylindrical stent is increased or reduced in width.

13. The covered stent of claim 2, wherein when a plurality of intervals at which no PTFE tape is provided on the inside of the cylindrical stent is provided, the intervals are different from each other in width.

14. The covered stent of claim 5, wherein when mounting the PTFE tape, the section at which the PTFE tape is provided on the inside of the cylindrical stent is increased or reduced in width.

15. The covered stent of claim 5, wherein when a plurality of sections at which the PTFE tape is provided on the inside of the cylindrical stent is provided, the sections are different from each other in width.

16. The method of claim 9, wherein the interval at which no PTFE tape is provided on the inside of the cylindrical stent is increased or reduced in width.

17. The method of claim 9, wherein when a plurality of the intervals at which no PTFE tape is provided on the inside of the cylindrical stent is provided, the intervals are different from each other in width.
